(19) United States
(12) Patent Application Publication
(10) Pub. No.: US 2009/0030279 A1
(43) Pub. Date: Jan. 29, 2009

(54) METHOD AND SYSTEM FOR MANAGING POWER CONSUMPTION IN A COMPACT DIAGNOSTIC CAPSULE

(76) Inventors: Dennis R. Zander, Penfield, NY (US); Peter Katevatis, Philadelphia, PA (US)

Correspondence Address: JAECKLE FLEISCHMANN & MUGEL, LLP, 190 Linden Oaks, ROCHESTER, NY, 14625-2812, US

(21) Appl. No.: 12/175,901

(22) Filed: Jul. 18, 2008

Related U.S. Application Data
(60) Provisional application No. 60/952,284, filed on Jul. 27, 2007.

(51) Int. Cl. A61B 1/00

(52) U.S. Cl. 600/118

(57) ABSTRACT

A diagnostic capsule has a sensor system, a transmitter, and a controller. The controller is configured to detect one or more target conditions external to the diagnostic capsule based on target data from the sensor system and enable the transmitter to transmit diagnostic data, wherein the diagnostic data is collected by the sensor system while the one or more target conditions are present. A diagnostic system utilizing the diagnostic capsule is also disclosed. A method of managing power consumption in a diagnostic capsule is also disclosed. A target sensor is enabled. The target sensor is checked for a target condition. At least one diagnostic capsule subsystem is enabled if the target condition is present. The target sensor is further checked for the target condition. The at least one diagnostic capsule subsystem is disabled if the target condition is not present.

### RELATED APPLICATIONS

This patent application claims priority to U.S. provisional patent application 60/952,284 filed on Jul. 27, 2007 and entitled, “METHOD AND SYSTEM FOR MANAGING POWER CONSUMPTION IN A COMPACT DIAGNOSTIC CAPSULE.” Accordingly, U.S. provisional patent application 60/952,284 is also hereby incorporated by reference in its entirety.

### FIELD

The claimed invention generally relates to compact diagnostic capsules, and more particularly to methods and systems for managing power consumption in a compact diagnostic capsule. The claimed invention further relates to the preferential transmission and recording of diagnostic data from a diagnostic capsule.

### BACKGROUND

Although great strides in cancer treatment have been developed over the years, cancer still remains among the leading causes of death in humans. One of the driving factors in our ability to successfully fight cancer is the ability to detect cancerous tissue at an early stage. Early detection requires regular check-ups and is also dependent on the ability of physicians to inspect a variety of areas on and within a patient's body, depending on the type of cancer being screened-for. While blood tests can be indicative of a cancerous condition within a person's body, they do not always determine the type of cancer and can not pin-point the exact location of the cancer. Therefore, a visual and/or imaging inspection is often more desirable, either on its own or in conjunction with other types of tests.

Visual and/or imaging inspections of portions of the gastro-intestinal (GI) tract have been made possible in the last century, for the determination of cancerous and other medical conditions, by using endoscope technology. An endoscope is a probe which is inserted either in the mouth or nose end of the alimentary canal or the anal end of the alimentary canal. A modern endoscope is fitted with an illumination source and a video camera or image sensor which can relay images of the areas it is manipulated into by a medical professional. The endoscopic probe is connected to an external monitor and/or image storage device by a cable. The probes are also manipulated and guided into place by an operator using the same or a different cable. While valuable, these types of endoscopic procedures risk tissue perforation and are uncomfortable for patients, often requiring the use of sedatives. Furthermore, there are still areas of the alimentary canal which can not be reached readily by an endoscope, simply because it is too difficult to manipulate the probe into certain highly twisted areas, such as the small intestine.

More recently, advances in micro-assembly and integration have made it possible to create endoscopic capsules which are small enough to be swallowed by a patient and which have no wires or cables connecting them the outside world. These endoscopic capsules wirelessly transmit image data to a receiver located outside of a patient's body as the endoscopic pill passes through the patient's body. One of the major limiting factors in how small endoscopic pills can be made is the space needed for on-board battery power in order to support the image capture and transmission needs of the pill as it passes through the entire gastrointestinal (GI) tract. Unfortunately, it can take up to seventy-two hours or longer for an endoscopic pill to pass through the GI tract; current battery technology does not support continuous video operations during that time frame in an ingestible pill-sized-package. For example, a product referred-to as SmartPill in an article entitled, “A Camera You Can Swallow” by Bridget Elton in the Spring 2006 edition of Electronics Education, pages 12-13, describes a pill which senses and records temperature, pressure, and pH within the GI tract for up to 72 hours by purposely leaving imaging capability out of the electronic pill in order to attain the desired battery life. While the data collected by this type of device may be helpful to medical professionals, it unfortunately can not provide images of the GI tract.

As a result, a variety of implementations of endoscopic capsules have been developed which accept and/or try to deal with the problem of limited battery life. In most endoscopic capsules, the battery is inserted or otherwise turned-on or activated just prior to asking a patient to swallow the capsule. In some instances, upon activation of the battery, a light source and imaging device on-board the pill begin continuous operation. Images are captured and transmitted to an external receiver for as long as the battery power is sufficient. Despite advances in battery technology, such devices typically run out of power before the endoscopic capsule reaches the lower gastro-intestinal tract, including the colon. Therefore, medical professionals may receive helpful information about only a part of the patient's alimentary canal. Considering that colon cancer is a leading cause of death among humans, solutions which ignore the colon are incomplete.

Competing endoscopic capsule designs have ways to turn on the endoscopic capsule a predetermined time after the capsule has been ingested. For example, U.S. Pat. No. 7,112,752 discloses a method of delaying the powering-on of an endoscopic capsule by using an insulative pH sensitive material to insulate normally-closed contacts on the capsule's power switch. Certain areas of the GI tract are known to have different pH ranges. For example, the pH in the stomach is from about 1-2, while the pH in the colon is typically above 7. A pH sensitive material may be chosen to dissolve under desired pH conditions and thereby allow the normally-closed switch to activate the endoscopic camera in a target area of the body. However, since this approach is targeted by region, it will inherently leave out other regions which may be cancerous or otherwise of-interest. Furthermore, although only a portion of the alimentary canal will be imaged, the health care professional using this method may still be facing tens of hours of video to review as the endoscopic capsule passes through the targeted regions.

In an alternate approach, a research paper entitled, “Lab-on-a-Chip Technology, as Remote Distributed Format for Disease Analysis”, written by Professor Jon Cooper of the University of Glasgow, was presented on Apr. 6^{th}, 2004 at the International Workshop of Wearable and Implantable Body Sensor Networks held by the Imperial College of London. The endoscopic capsule disclosed in this paper may be equipped with a receiver to allow remote control over the capsule's function, switching sensors and/or power on and off on-demand. Unfortunately, this approach may require continuous intervention by a skilled medical professional in order to provide any possible power savings. The patient also has to remain within range of the medical professional to achieve such savings, and this can be impractical and/or inconvenient during the 72 or more hours it can take for the endoscopic capsule to traverse the GI tract.

In order to attempt to allow endoscopic capsules to transmit more image data without expending additional battery power, certain capsules are outfitted with image compression capabilities. For example, the endoscopic capsule disclosed in published U.S. Patent Application Publication 2006/0262186 employs a compression algorithm or a compression circuit to minimize the size of each image captured by the capsule; this can reduce the transmission load on the capsule. While this type of capsule may have reduced demands on the battery carried by the capsule, there is no indication that the battery will be able to last up to 72 hours or longer for a trip through the entire GI tract. While battery power may be conserved, the method does not preferentially identify regions of diagnostic interest; a medical professional is required to search through the entire video record to find any areas of interest.

Another approach to assist endoscopic capsules in conserving battery power is outlined in a white paper entitled, “The Ultra Low-Power Wireless Medical Device Revolution,” by Peter Bradley which was published in the April 2005 edition of Medical Electronics Manufacturing. The white paper outlines a duty-cycling strategy used by some endoscopic capsules to minimize their power usage. Under duty cycling, rather than transmitting constantly, the capsule transmitter transmits on a regular interval. Duty cycling can be electronically imposed with an on-board clock or timer. Duty-cycling may also be artificially imposed using the regular and naturally occurring peristaltic motion found in the GI tract as disclosed in U.S. Pat. No. 5,604,531 where the endoscopic capsule is made pressure sensitive and is designed to sense the contractions of the muscles within the GI tract and transmit every time there is a contraction.

The use of duty cycling can lead to several different situations when images are being captured by the endoscopic capsule. In a first scenario, the image data may be collected continuously, and the capsule may have enough on-board capacity to buffer the data until the transmission duty cycle interval arrives. At this point, the device may have a high-enough data transmission rate during the transmit portion of the duty cycle to completely empty the stored image buffer. The net effect in this first scenario is that a complete picture of the endoscopic capsule's journey through the alimentary canal may be stitched together from the duty-cycle transmission bursts. Realistically, however, such a device would have to have a large amount of on-board memory which can take up space, increase the cost of an essentially disposable device, and in the end, the medical professional still has to examine the entire video record of the capsule's journey through the GI tract. In a second scenario, the capsule's ability to collect image data may exceed the bandwidth of the duty-cycle transmission window such that regular portions of the image data are not able to be transmitted from the explorer. Unfortunately, this results in an incomplete picture of the alimentary canal, and the potential for areas of interest to be missed or not completely imaged.

In addition to duty cycling, a further approach to preserving battery life on-board an ingestible electronic pill is described in a paper entitled, “Implementation of Radiotelemetry in a Lab-in-a-Pill Format,” authored by Erik A. Johannessen, et al., as published in Lab Chip, 2006, volume 6, pages 39-45. (First published as an Advance Article on the web Nov. 21, 2005.) The electronic pill, which is the focus of the article, has multiple sensor inputs for measurements such as pH, temperature, and pressure. An analytical signal for one or more of the measurements is compared to the previous signals at a sample interval of 1 second. When the difference between the most recent measurement and the previous measurement exceeds a pre-determined threshold, the pill transmits the data. When no change is observed between successive measured values, then no transmission occurs. If the measurements are relatively static within the alimentary canal, then this type of approach has the potential to reduce power consumption by reducing data transmissions. Unfortunately, this type of system, when applied to an endoscopic capsule has several shortcomings. Areas which may be of interest may transition gradually from neighboring benign or normal areas and therefore the comparative technique described by this reference may miss slow spatial variations in tissue state that may be associated with disease states. Furthermore, while such a comparison algorithm will preferentially identify well-defined edges of differentiated tissue, it will tend to measure no further information about the differential tissue itself. As the capsule continues to pass by the tissue of interest, the comparison algorithm will conceivably show no change, so no further images will be generated at this point. When the capsule completes its pass by the tissue of interest, the comparison algorithm will show a change and send a second image. Unfortunately, no intervening imagery will be obtained between the start of the tissue of interest and the end of the tissue of interest. As a result of incomplete imagery, a patient has the potential to be mis-diagnosed or may face follow-up and possibly more invasive endoscopy techniques to obtain the desired imagery for diagnosis.

Therefore, there is a need for a less expensive, space-saving, power saving diagnostic capsule, such as an endoscopic capsule, that is not hampered by the shortcomings outlined above. The diagnostic capsule will also preferably help reduce the amount of time patients need to spend in a medical facility and reduce the amount of medical professional time needed to assist with and analyze the data from the diagnostic capsule.

### SUMMARY

A diagnostic capsule is disclosed. The diagnostic capsule comprises a sensor system, a transmitter, and a controller. The controller is configured to detect one or more target conditions external to the diagnostic capsule based on target data from the sensor system and to enable the transmitter to transmit diagnostic data, wherein the diagnostic data are collected by the sensor system while the one or more target conditions are present.

A diagnostic system is also disclosed. The diagnostic system comprises a diagnostic capsule. The diagnostic capsule further comprises a sensor system, a transmitter, and a controller. The controller is configured to detect one or more target conditions external to the diagnostic capsule based on target data from the sensor system and to enable the transmitter to transmit diagnostic data, wherein the diagnostic data are collected by the sensor system while the one or more target conditions are present. The diagnostic system also has at least one receiver configured to receive transmissions from the transmitter. The diagnostic system further includes a receiver controller coupled to the at least one receiver. The receiver controller is configured to store transmitted diagnostic data received at the at least one receiver from the diagnostic capsule.

A method of managing power consumption in a diagnostic capsule is also disclosed. This method comprises a number of steps including, enabling a target sensor, and checking the target sensor for a target condition. At least one diagnostic capsule subsystem is enabled if a target condition is present. The target sensor is further checked for a target condition. The at least one diagnostic capsule subsystem is disabled if the target condition is not present.

### BRIEF DESCRIPTION OF THE DRAWINGS

FIGS. 1-3 schematically illustrate different embodiments of a diagnostic capsule.

FIG. 4 schematically illustrates an embodiment of a diagnostic system utilizing an embodiment of a diagnostic capsule.

FIG. 5 illustrates one embodiment of a method for managing power consumption in a diagnostic capsule.

FIG. 6A illustrates another embodiment of a method for managing power consumption in a diagnostic capsule.

FIG. 6B illustrates one possible timing diagram which may result from the embodied method for managing power consumption in a diagnostic capsule which is illustrated in FIG. 6A.

FIG. 7A illustrates another embodiment of a method for managing power consumption in a diagnostic capsule.

FIG. 7B illustrates one possible timing diagram which may result from the embodied method for managing power consumption in a diagnostic capsule which is illustrated in FIG. 7A.

FIG. 8A illustrates another embodiment of a method for managing power consumption in a diagnostic capsule.

FIG. 8B illustrates one possible timing diagram which may result from the embodied method for managing power consumption in a diagnostic capsule which is illustrated in FIG. 8A.

FIG. 9A illustrates another embodiment of a method for managing power consumption in a diagnostic capsule.

FIG. 9B illustrates one possible timing diagram which may result from the embodied method for managing power consumption in a diagnostic capsule which is illustrated in FIG. 9A.

FIG. 10A illustrates another embodiment of a method for managing power consumption in a diagnostic capsule.

FIG. 10B illustrates one possible timing diagram which may result from the embodied method for managing power consumption in a diagnostic capsule which is illustrated in FIG. 10A.

FIG. 10C illustrates another possible timing diagram which may result from the embodied method for managing power consumption in a diagnostic capsule which is illustrated in FIG. 10A.

FIGS. 11-12 schematically illustrate alternate embodiments of diagnostic systems using embodiments of a diagnostic capsule.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

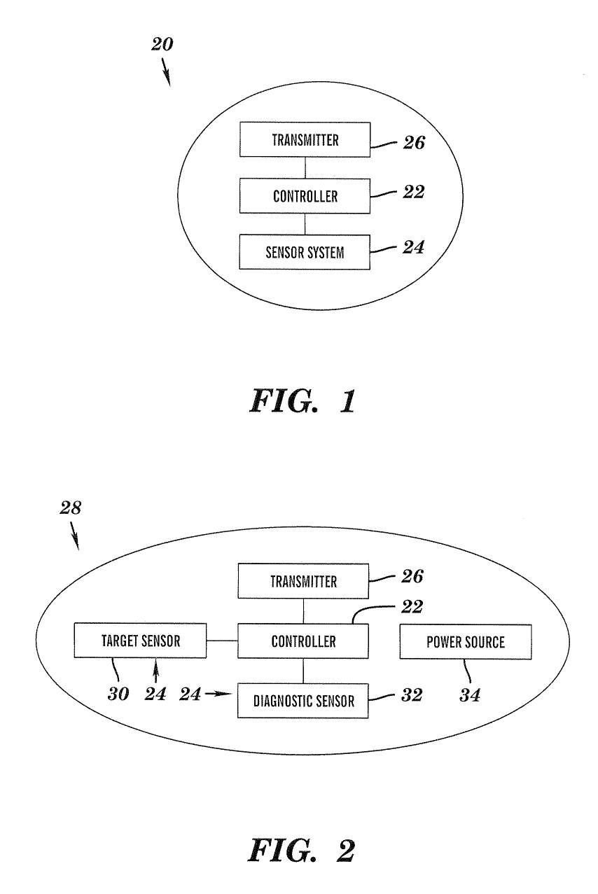

FIG. 1 schematically illustrates an embodiment of a diagnostic capsule 20. The diagnostic capsule 20 may be designed for human or animal use and as such is preferably sized to pass through the alimentary canal with little or no discomfort. The shape of the diagnostic capsule in the drawings is only for illustration's sake. It should be understood that other shapes of diagnostic capsules are intended to be covered by the scope of this specification. One example of a type of diagnostic capsule 20 which may be designed for human use is an endoscopic capsule. In other applications, the diagnostic capsule may be designed for industrial or agricultural use, for example in the inspection of pipes or pipelines. Other uses for a diagnostic capsule will be apparent to those skilled in the art and are intended to be covered by the scope of the appended claims. For convenience, the diagnostic capsule 20 will be described with regard to testing of the alimentary canal.

The diagnostic capsule 20 has a controller 22. Depending on the embodiment, the controller 22 may be any type of computer, microprocessor, distributed processors, parallel processors, application specific integrated circuit (ASIC), digital components, analog electrical components, and/or any combination thereof. The controller 22 can include a memory for storing executable instructions as well as data. The memory may be volatile and/or non-volatile. The controller 22 is coupled to a sensor system 24 which can include one or more types of sensors for gathering data about the environment in which the diagnostic capsule 20 will operate. Different types of sensors which may be used in the sensor system 24 include, but are not limited to a pH sensor, a temperature sensor, a pressure sensor, a biological sensor, a bacterial sensor, a protein sensor, a chemical sensor, a light sensor, a spectral sensor, a radiation sensor, and an imaging sensor.

The sensor system 24 is configured to selectively collect target data and diagnostic data when enabled by the controller 22. Target data is used by the controller 22 to decide if a target condition exists, for example a tissue anomaly among otherwise healthy tissue. Diagnostic data is data gathered in the area in which the target condition exists. In some embodiments of a diagnostic capsule 20, the target data and the diagnostic data may come from the same sensor in the sensor subsystem 24. In other embodiments, the target data and the diagnostic data will come from different sensors in the sensor subsystem 24. For example, a spectral sensor could be used for the target data. When a target spectral response is noted, the controller 22 would determine that a target condition exists. An imaging or video sensor could be used to gather diagnostic data corresponding to the time when the target condition exists as indicated by the target data.

The controller 22 is also coupled to a transmitter 26. Various types of transmitters may be selected for transmitter 26, for example, but not limited to an ultra-low power RF transmitter. The transmitter 26 may be enabled by the controller 22 to transmit at least the diagnostic data collected by the sensor system 24. As pointed out in the background section above, however, the power consumed by both the sensor system 24 (especially when image diagnostic data is being collected) and by the transmitter 26 may be too much for the power source (not shown in FIG. 1), which is coupled to the diagnostic capsule 20, for the diagnostic capsule 20 to gather data during its entire intended reconnaissance path. Therefore, in order to help reduce the power requirements of the diagnostic capsule 20, the controller 22 in this embodiment may be configured to detect one or more target conditions external to the diagnostic capsule 20 based on target data from the sensor system 24. The controller 22 can selectively enable the collection of diagnostic data and/or the transmission of diagnostic data only when one or more target conditions are present (as determined from an analysis of the target data). Using this configuration, the diagnostic capsule 20 is able to monitor the target data continuously so that all areas of interest have a chance to be explored. However, since there will likely be few target condition events, the power requirements may be greatly reduced by only turning on the transmitter 26 and/or the diagnostic data sensor as determined to be necessary based on the target data.

FIG. 2 schematically illustrates another embodiment of a diagnostic capsule 28. Although in some embodiments the target data and the diagnostic data may come from the same sensor in the sensor system 24, in the embodiment of FIG. 2, sensor system 24 has a target sensor 30 which may be enabled by the controller 22 to gather target data; and the sensor system 24 also has a diagnostic sensor 32 which may be enabled by the controller 22 to gather diagnostic data. The diagnostic capsule 28 is self-contained, and therefore relies on a limited power source 34 while in operation. As in the embodiment of FIG. 1, the controller 22 in the embodiment of FIG. 2 may be configured to selectively enable the transmitter 26 and/or the diagnostic sensor 32 based on the target data from an enabled target sensor 30. In some embodiments, the target sensor 30 may always be enabled either by controller 22 default instruction or by hardwiring to the target sensor 30.

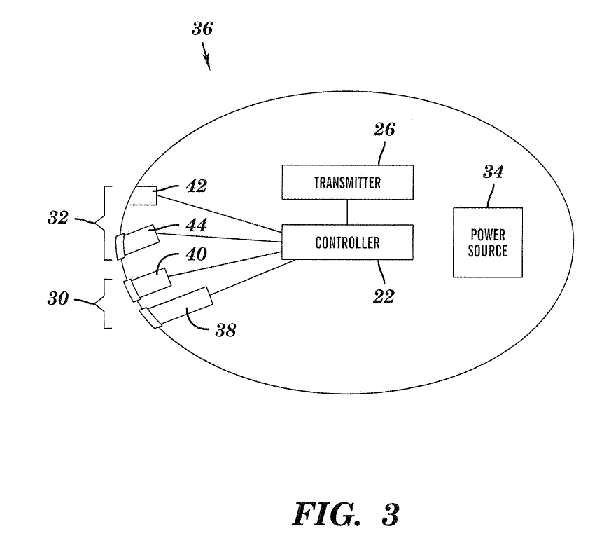

FIG. 3 schematically illustrates a further embodiment of a diagnostic capsule 36. In this embodiment, the target sensor 30 includes a light source 38, such as a laser, an LED, or other light emitting material, which works in conjunction with a spectral sensor 40. Therefore, the target sensor 30 in this embodiment is configured as a spectral imaging sensor. Although other types of sensors may be used as a target sensor 30, depending on the application, in the area of cancer detection, for example, spectral imaging has shown great promise in being able to differentiate healthy tissue from cancerous or pre-cancerous tissue. Various spectral imaging techniques are known to those skilled in the art, such as native fluorescent spectroscopy, Raman spectroscopy, spectral wing analysis in the far-red to near-infrared spectrum, and diffuse reflectance spectroscopy employing the Kubelka-Munk function. In embodiments such as FIG. 3, the target sensor 30 may be configured to utilize these types of spectral imaging techniques and similar or equivalent techniques. Embodiments utilizing spectral imaging techniques to generate target data are exploiting the inherent differences in the chemical composition of normal, benign, and cancerous tissues for a given tissue type. These differences in cellular composition result in distinct spectral profiles, which, in turn, make it possible for the controller 22 to compare the spectral profile of an unknown tissue sample to those of known normal, benign, and cancerous tissues in order to determine whether a target condition exists.

The diagnostic sensor 32 of the diagnostic capsule 36 is an image sensor having a light source 42 for illuminating the environment where images will be captured and a micro camera 44 for capturing one or more images (diagnostic data) of the environment when enabled by controller 22. As in the embodiment of FIG. 1, the controller 22 in FIG. 3 may be configured to selectively enable the transmitter 26 and/or the diagnostic sensor 32 based on whether a target condition is indicated from the target data supplied by target sensor 30.

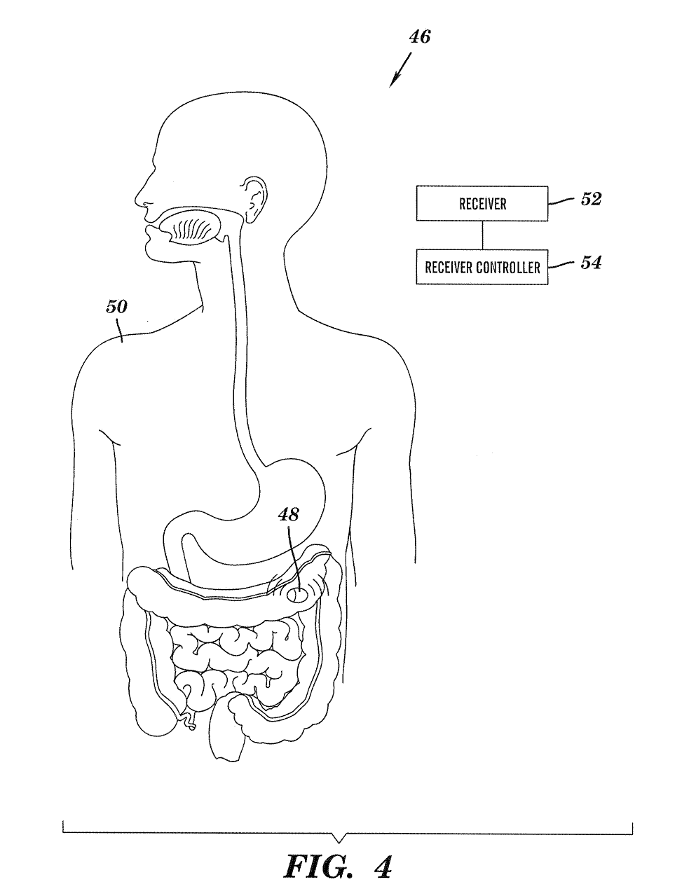

Since the embodied diagnostic capsules 20, 28, 36 will be transmitting at least diagnostic data, they will preferably be used as part of a diagnostic system which is capable of receiving the transmitted data. FIG. 4 schematically illustrates one embodiment of a diagnostic system 46 which can be used in applications where a diagnostic capsule 48 is ingested by a subject 50 and moves through their alimentary canal. The diagnostic system 46 has a receiver 52 coupled to a receiver controller 54, both of which are located external to the subject 50. Since receiver 52 is configured to receive transmissions from diagnostic capsule 48, the receiver 52 should be positioned within transmission range of the diagnostic capsule 48. The receiver controller 54 may be configured to store the transmitted diagnostic data received at the receiver 52 from the diagnostic capsule 48. The receiver 52, and optionally the receiver controller 54 may be portable and even wearable so that the subject 50 may carry the receiver 52 with them for increased freedom of movement during the relatively long time it can take a diagnostic capsule to move through the alimentary canal.

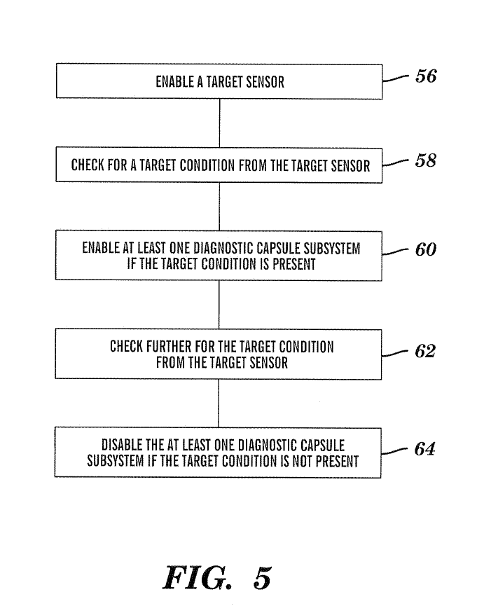

FIG. 5 illustrates one embodiment of a method for managing power consumption in a diagnostic capsule. A target sensor is enabled 56. Examples of target sensors may include, but are not limited to pH sensors, temperature sensors, pressure sensors, biological sensors, bacterial sensors, protein sensors, chemical sensors, light sensors, spectral sensors, radiation sensors, and imaging sensors. Preferably the target sensor which is enabled 56 is a low power consumption device since it has the potential to be on a relatively long time. In some embodiments, however, the target sensor may not consume low levels of power. Next, the target sensor is checked 58 to see if a target condition exists. For example, if the target sensor is a spectral sensor, a target condition may be defined to exist when the spectral response from light reflected off of illuminated tissue correlates to a known spectral response for cancerous tissue. If a target condition is present, at least one diagnostic capsule subsystem is enabled 60. Suitable diagnostic capsule subsystems which may be enabled include a transmitter and/or a diagnostic sensor. Next the target sensor is checked again 62 to see if the target condition still exists. If the target condition no longer exists, then the at least one diagnostic capsule subsystem is disabled 64.

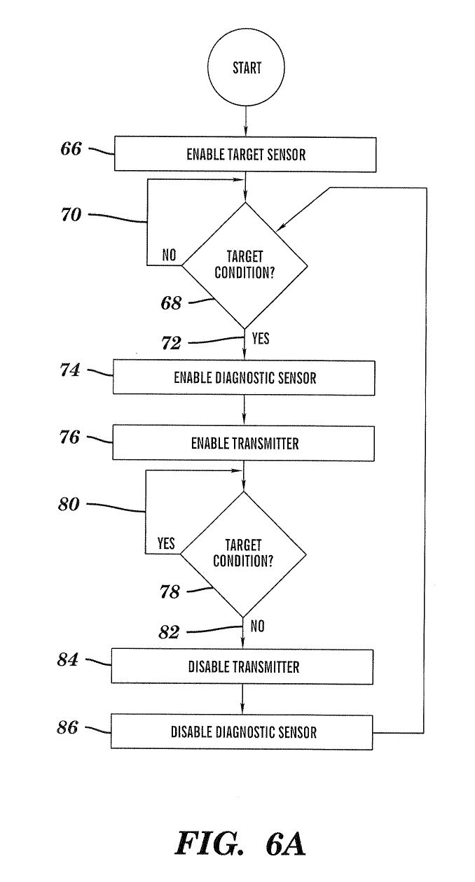

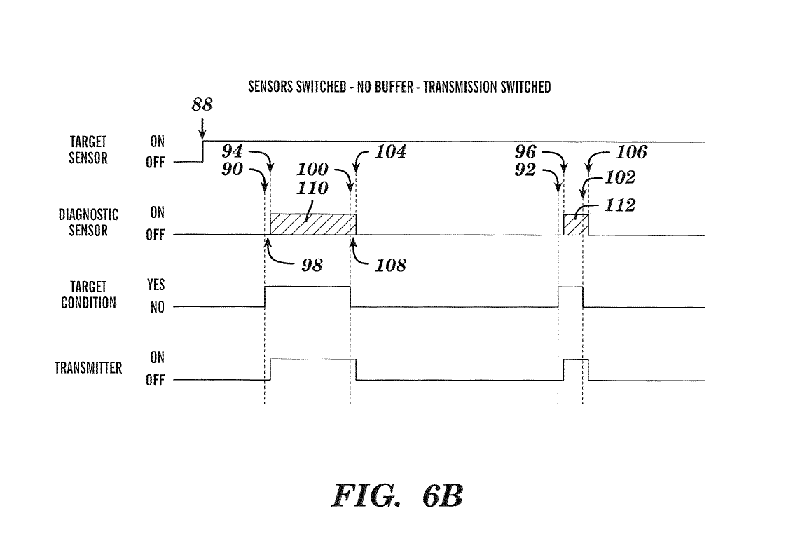

FIG. 6A illustrates another embodiment of a method for managing power consumption in a diagnostic capsule. A target sensor is enabled 66 and the target sensor is checked 68 to see if a target condition exists. If no target condition exists 70, then the existence of a target condition continues to be checked 68. If a target condition exists 72, a diagnostic sensor is enabled 74 and a transmitter is enabled 76. The enabled transmitter can transmit at least the data gathered by the enabled diagnostic sensor while it is enabled. Since the transmitter and the diagnostic sensor are not enabled all of the time, significant power requirement reductions may be realized by the diagnostic capsule. The target sensor is checked again 78 to see if the target condition persists. If the target condition still exists 80, then the existence of the target condition continues to be checked 78 while the diagnostic sensor and the transmitter remain enabled. If the target condition ends 82, then the transmitter is disabled 84 and the diagnostic sensor is disabled 86.

FIG. 6B illustrates one possible timing diagram which may result from the embodied method for managing power consumption in a diagnostic capsule which is illustrated in FIG. 6A. First, the target sensor is switched on 88. The controller monitors the output from the target sensor to determine if a target condition exists. In the illustrated diagram, a transition from no target condition to a target condition occurs at times 90 and 92. After a target condition occurs 90, 92 the diagnostic sensor and the transmitter are turned on at times 94 and 96, respectively. As can be seen from the diagram, there may be a small delay 98 from existence of the target condition to enablement of the diagnostic sensor and the transmitter. In this example, a transition from a target condition to no target condition occurs at times 100 and 102. After the target condition goes away 100, 102 the diagnostic sensor and the transmitter are turned off at times 104 and 106 respectively. There may be a small delay 108 following the transition to no target condition before the diagnostic sensor and the transmitter are disabled. In this embodiment, the diagnostic sensor is enabled at substantially the same time as the transmitter and only for a target condition. Since no data buffer is used in this embodiment, the diagnostic data available for transmission is shown in shaded areas 110 and 112.

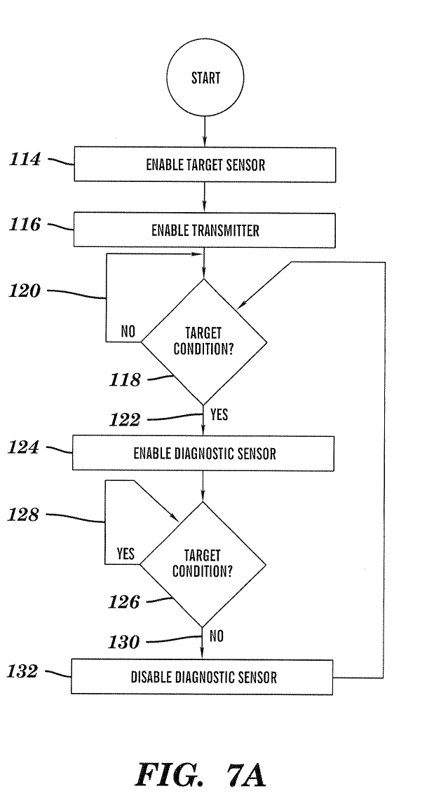

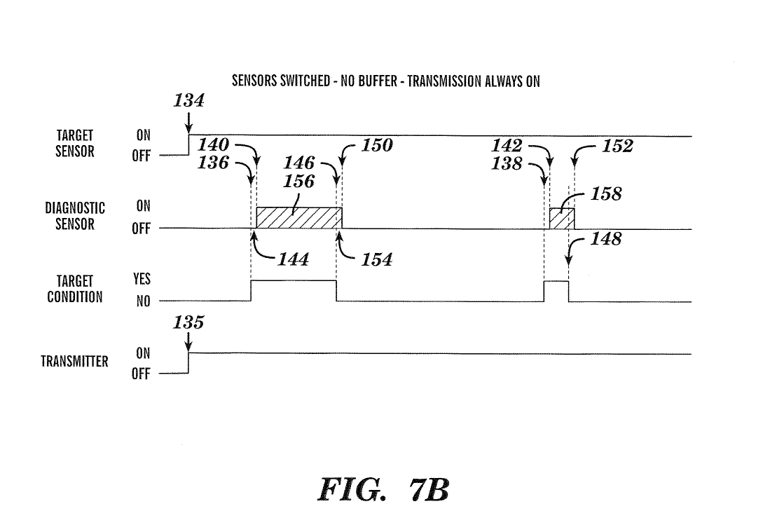

FIG. 7A illustrates another embodiment of a method for managing power consumption in a diagnostic capsule. A target sensor is enabled 114, a transmitter is enabled 116, and the target sensor is checked 118 to see if a target condition exists. If no target condition exists 120, then the existence of a target condition continues to be checked 118. If a target condition exists 122, a diagnostic sensor is enabled 124. The transmitter, which was already enabled can transmit at least the data gathered by diagnostic sensor while it remains enabled. Since the diagnostic sensor is not enabled all of the time, significant power requirement reductions may be realized by the diagnostic capsule, while still being able to maintain transmissions, for example for telemetry and capsule location purposes. The target sensor is checked again 126 to see if the target condition persists. If the target condition still exists 128, then the existence of the target condition continues to be checked 126 while the diagnostic sensor remains enabled. If the target condition ends 130, then the diagnostic sensor is disabled 132.

FIG. 7B illustrates one possible timing diagram which may result from the embodied method for managing power consumption in a diagnostic capsule which is illustrated in FIG. 7A. First, the target sensor is switched on 134 and the transmitter is also switched on 135. The controller monitors the output from the target sensor to determine if a target condition exists. In the illustrated diagram, a transition from no target condition to a target condition occurs at times 136 and 138. After a target condition occurs 136, 138 the diagnostic sensor is turned on at times 140 and 142, respectively. As can be seen from the diagram, there may be a small delay 144 from existence of the target condition to enablement of the diagnostic sensor. In this example, a transition from a target condition to no target condition occurs at times 146 and 148. After the target condition goes away 146, 148 the diagnostic sensor is turned off at times 150 and 152, respectively. There may be a small delay 154 following the transition to no target condition before the diagnostic sensor is disabled. In this embodiment, the diagnostic sensor is substantially enabled only for a target condition. Since no data buffer is used in this embodiment, the diagnostic data available for transmission is shown in shaded areas 156 and 158.

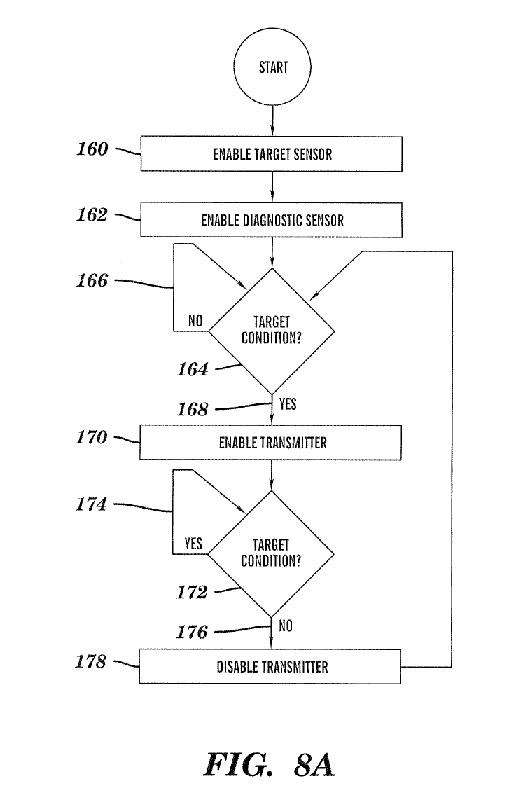

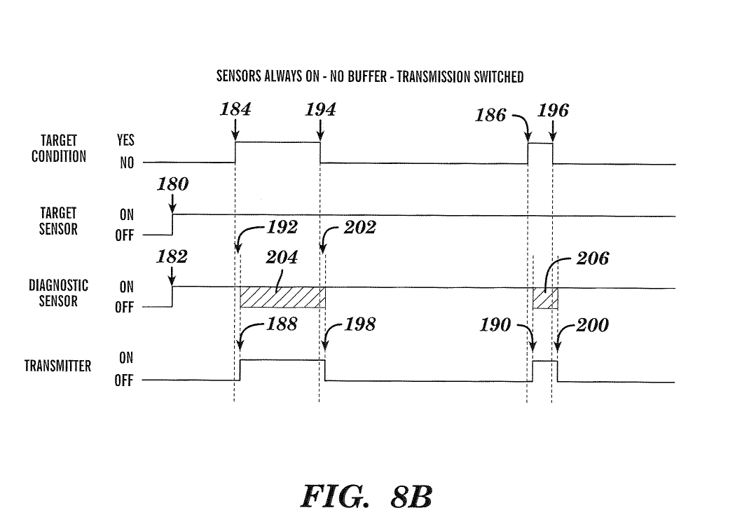

FIG. 8A illustrates another embodiment of a method for managing power consumption in a diagnostic capsule. A target sensor is enabled 160, a diagnostic sensor is enabled 162, and the target sensor is checked 164 to see if a target condition exists. If no target condition exists 166, then the existence of a target condition continues to be checked 164. If a target condition exists 168, a transmitter is enabled 170. The enabled transmitter can transmit at least the data gathered by the diagnostic sensor. Since the transmitter is not enabled all of the time, significant power requirement reductions may be realized by the diagnostic capsule. The target sensor is checked again 172 to see if the target condition persists. If the target condition still exists 174, then the existence of the target condition continues to be checked 172 while the transmitter remains enabled. If the target condition ends 176, then the transmitter is disabled 178.

FIG. 8B illustrates one possible timing diagram which may result from the embodied method for managing power consumption in a diagnostic capsule which is illustrated in FIG. 8A. First, the target sensor is switched on 180 and the diagnostic sensor is switched on 182. The controller monitors the output from the target sensor to determine if a target condition exists. In the illustrated diagram, a transition from no target condition to a target condition occurs at times 184 and 186. After a target condition occurs 184, 186 the transmitter is turned on at times 188 and 190, respectively. As can be seen from the diagram, there may be a small delay 192 from existence of the target condition to enablement of the transmitter. In this example, a transition from a target condition to no target condition occurs at times 194 and 196. After the target condition goes away 194, 196 the transmitter is turned off at times 198 and 200, respectively. There may be a small delay 202 following the transition to no target condition before the transmitter is disabled. In this embodiment, the transmitter is substantially only enabled when there is a target condition. Since no data buffer is used in this embodiment, the diagnostic data available for transmission is shown in shaded areas 204 and 206.

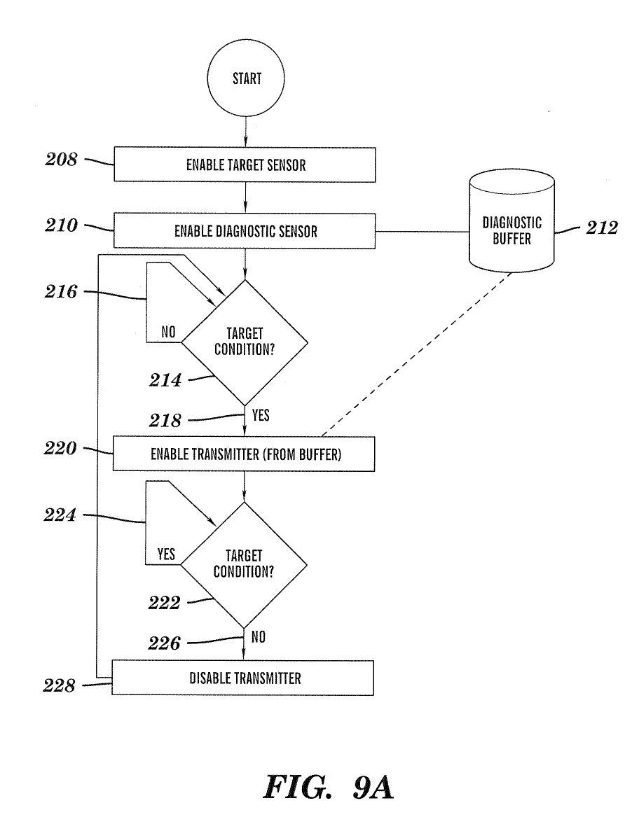

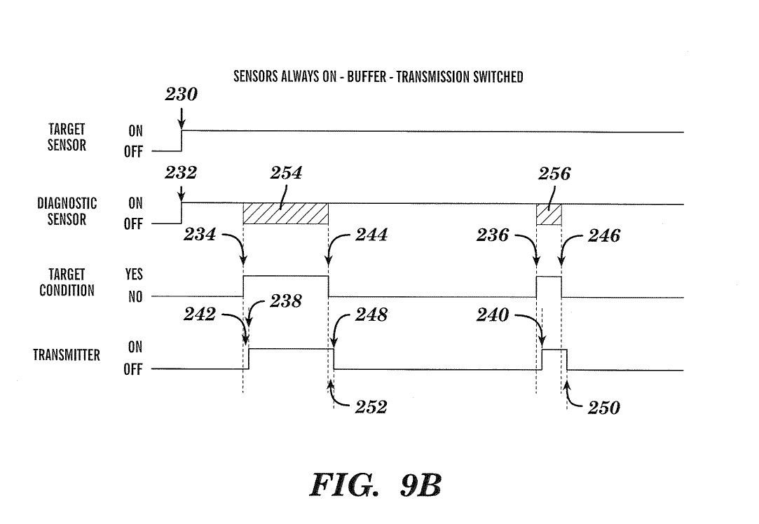

FIG. 9A illustrates another embodiment of a method for managing power consumption in a diagnostic capsule. A target sensor is enabled 208 and a diagnostic sensor is enabled 210. A buffer is provided 212 for the diagnostic data. The buffer can be a first-in-first-out device such as a shift register. Alternatively, the buffer can be implemented by writing diagnostic data to changing positions in a memory by managing pointers to the current and buffered memory locations. Other types of buffers are known to those skilled in the art and are also intended to be included. The target sensor is checked 214 to see if a target condition exists. If no target condition exists 216, then the existence of a target condition continues to be checked 214. If a target condition exists 218, a transmitter is enabled 220. The enabled transmitter can transmit data gathered by the buffer from the diagnostic sensor. Since the transmitter is not enabled all of the time, significant power requirement reductions may be realized by the diagnostic capsule. The target sensor is checked again 222 to see if the target condition persists. If the target condition still exists 224, then the existence of the target condition continues to be checked 222 while the transmitter remains enabled. If the target condition ends 226, then the transmitter is disabled 228.

FIG. 9B illustrates one possible timing diagram which may result from the embodied method for managing power consumption in a diagnostic capsule which is illustrated in FIG. 9A. First, the target sensor is switched on 230 and the diagnostic sensor is switched on 232. The controller monitors the output from the target sensor to determine if a target condition exists. In the illustrated diagram, a transition from no target condition to a target condition occurs at times 234 and 236. After a target condition occurs 234, 236, the transmitter is turned on at times 238 and 240, respectively. As can be seen from the diagram, there may be a small delay 242 from the existence of the target condition to enablement of the transmitter. In this example, a transition from a target condition to no target condition occurs at times 244 and 246. After the target condition goes away 244, 246 the transmitter is turned off at times 248 and 250, respectively. There may be a small delay 252 following the transition to no target condition before the transmitter is disabled. In this embodiment the transmitter is enabled substantially at the same time as the target condition, however any delays between sensing the target condition and enabling the transmitter can be compensated-for by choosing to transmit data from the buffer which corresponds to the actual sensing of the target condition. In other embodiments, it may also be desirable to use the buffer to transmit some of the diagnostic data from a time before the target condition, the data during the target condition, and some of the diagnostic date from a time after the target condition. This transitional data may be helpful to professionals analyzing the data from the diagnostic sensor. For this embodiment, the shaded areas 254 and 256 represent the diagnostic data which could be transmitted by the transmitter. It should be noted that the transmitted data does not have to line up in time with the transmitter enablement because a buffer is being used.

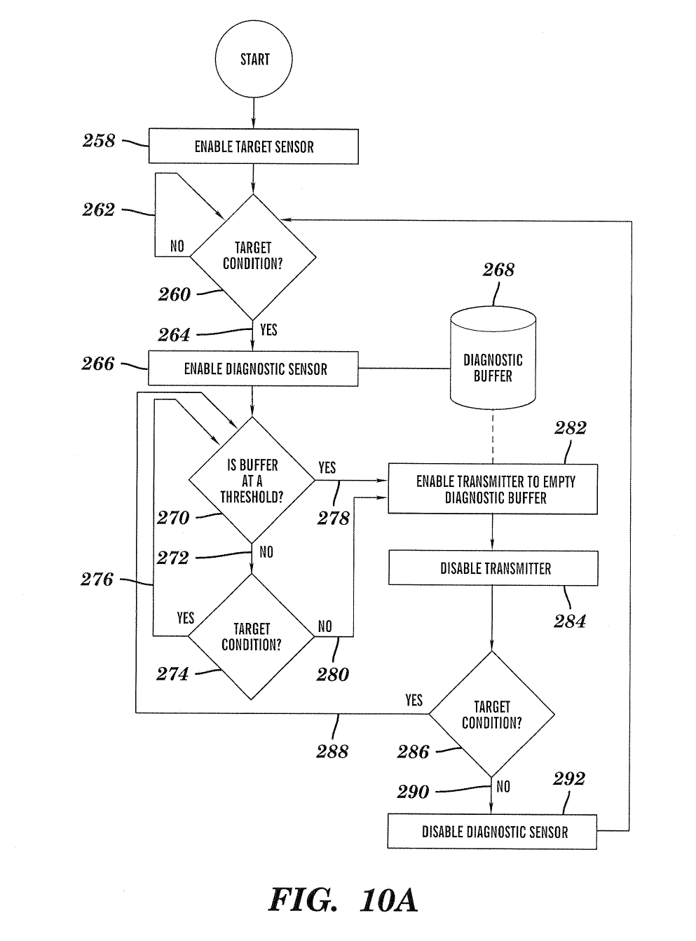

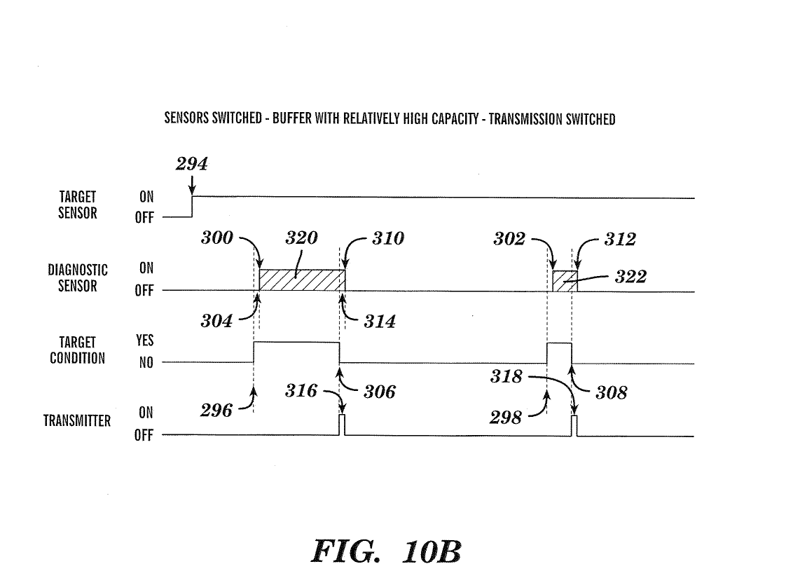

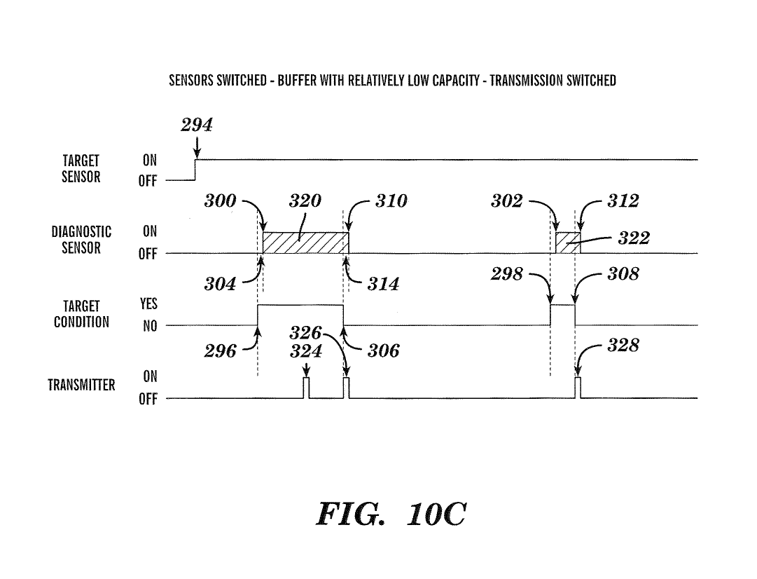

FIG. 10A illustrates another embodiment of a method for managing power consumption in a diagnostic capsule. A target sensor is enabled 258 and the target sensor is checked 260 to see if a target condition exists. If no target condition exists 262, then the existence of a target condition continues to be checked 260. If a target condition exists 264, a diagnostic sensor is enabled 266. A buffer is provided 268 for the diagnostic data. The buffer can be a first-in-first-out device such as a shift register. Alternatively, the buffer can be implemented by writing diagnostic data to changing positions in a memory by managing pointers to the current and buffered memory locations. Other types of buffers are known to those skilled in the art and are also intended to be included. The buffer is checked 270 to see if it is at a threshold. The threshold can be reached due to the buffer filling up or due to the buffer reaching a user-adjustable or design-adjustable limit for buffer size. If the buffer is not at a threshold 272, then the process re-checks 274 to see if the target condition still exists. If the target condition still exists 276, the buffer check 270 is repeated. If either the buffer check says the buffer is at a threshold 278 or the target condition re-check says the target condition no longer exists 280, then the transmitter is enabled 282 to empty the diagnostic buffer by transmitting the buffered diagnostic data which was stored by the buffer. The transmitter is then disabled 284. A final check 286 is performed to see if a target condition still exists. If the target condition still exists 288 the process goes back to the buffer check 270, since the previous data transmission to empty the buffer was likely due to the buffer having been full. New data is still being gathered in the recently emptied buffer. If the target condition does not exist 290, then the diagnostic sensor is disabled 292.

FIG. 10B illustrates one possible timing diagram which may result from the embodied method for managing power consumption in a diagnostic capsule which is illustrated in FIG. 10A. First, the target sensor is switched on 294. The controller monitors the output of the target sensor to determine if a target condition exists. In the illustrated diagram, a transition from no target condition to a target condition occurs at times 296 and 298. After a target condition occurs 296, 298, the diagnostic sensor is turned on at times 300 and 302, respectively. As can be seen from the diagram, there may be a small delay 304 from existence of the target condition to enablement of the diagnostic sensor. In this example, a transition from a target condition to a no target condition occurs at times 306 and 308. After the target condition goes away 306, 308 the diagnostic sensor is turned off at times 310 and 312, respectively. There may be a small delay 314 following the transition to no target condition before the diagnostic sensor is disabled. In this embodiment, the buffer was large enough to hold the diagnostic sensor data during each target condition without having to force a transmission for the buffer having reached its threshold. Transmissions 316 and 318 were triggered by the ending of separate target conditions. In this embodiment, the transmitter also has a relatively high throughput as evidenced by the comparatively small duration of the transmissions 316, 318 compared to their respective transmitted data regions 320 and 322.

FIG. 10C illustrates another possible timing diagram which may result from the embodied method for managing power consumption in a diagnostic capsule which is illustrated in FIG. 10A. The timing lines for the target sensor, the diagnostic sensor, and the target condition in FIG. 10C are identical to those in FIG. 10B, so the reference numerals are repeated and the discussion above is referred to with respect to those items. Where the embodiment of FIG. 10C differs from the embodiment of FIG. 10B is in the transmitter timing line. In this embodiment, the buffer filled up during the first target condition before the target condition ended. This resulted in a transmission emptying of the buffer 324 during the first target condition before the end of the target condition triggered another transmission emptying of the buffer 326. The second target condition was short enough in this embodiment that the buffer was able to handle the entire diagnostic sensor data during the target condition, and then the end of the second target condition triggered the final transmission of the buffer data 328.

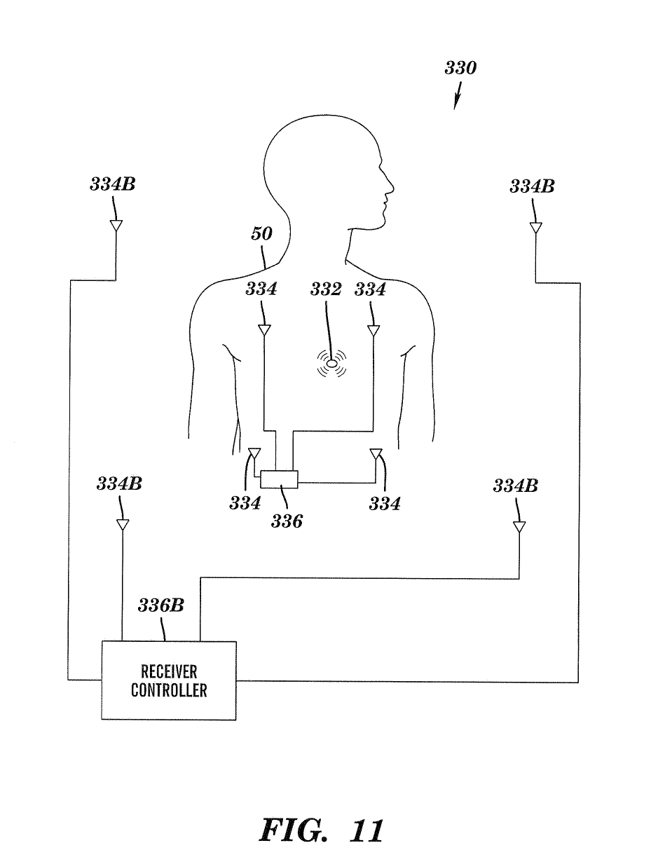

FIG. 11 schematically illustrates another embodiment of a diagnostic system 330 which can be used in applications where a diagnostic capsule 332 is ingested by a subject 50 and moves through their alimentary canal. The embodied diagnostic system 330 has at least three receivers 334 coupled to a receiver controller 336, all of which are located external to the subject 50. Since receivers 334 are configured to receive transmissions from diagnostic capsule 332, the receivers 334 should be positioned within transmission range of the diagnostic capsule 332. The receiver controller 336 may be configured to store the transmitted diagnostic data received at the receivers 334 from the diagnostic capsule 332. The receivers 334, and optionally the receiver controller 336 may be portable and even wearable so that the subject 50 may carry the receivers 334 with them for increased freedom of movement during the relatively long time it can take a diagnostic capsule to move through the alimentary canal. Receivers 334 and receiver controller 336 are illustrated in this embodiment as being worn by and/or attached to subject 50.

An alternate arrangement for the receivers and receiver controller is also illustrated in FIG. 11. The alternate receivers and receiver controller are labeled 334B and 336B, respectively. Alternate receivers 334B and alternate receiver controller 336B are not worn by or attached to subject 50. It should be understood that only one set of receivers/receiver controller (either 334 & 336 or 334B & 336B) is needed for the diagnostic system 330. For convenience, only the receivers 334 and receiver controller 336 will be discussed further.

The at least three receivers 334 are located in separate locations so that the receiver controller 336 may be configured to determine a position of the diagnostic capsule 332 relative to the at least three receivers 334 based on RF telemetry when the diagnostic capsule 332 is transmitting data. The receiver controller 336 may also be configured to store the determined location of the diagnostic capsule 332 relative to the at least three receivers 334 for one or more diagnostic data transmissions received from the transmitter. Furthermore, if the location of the test subject 50 relative to the at least three receivers 334 is known by the receiver controller 336, then the receiver controller 336 may be configured to determine a location of the diagnostic capsule 332 within the subject 50 based on the RF telemetry determination and the relationship between the subject 50 being tested and the at least three receivers 334. Such a diagnostic system 330 has the advantage that only data corresponding to target condition time-frames is being transmitted and stored so that medical professionals do not have to hunt through hours of images and video for pertinent data. Furthermore, the system 330 enables management of the power requirements on-board the diagnostic capsule 332 so that the entire alimentary canal may be examined, and substantially complete or entirely complete images or other data corresponding to the target regions is captured without gaps. Furthermore, positional information corresponding to the data being stored by the receiver controller 336 can be available in some embodiments to assist medical professionals in physically reaching target condition areas via follow-up surgery or similar procedures.

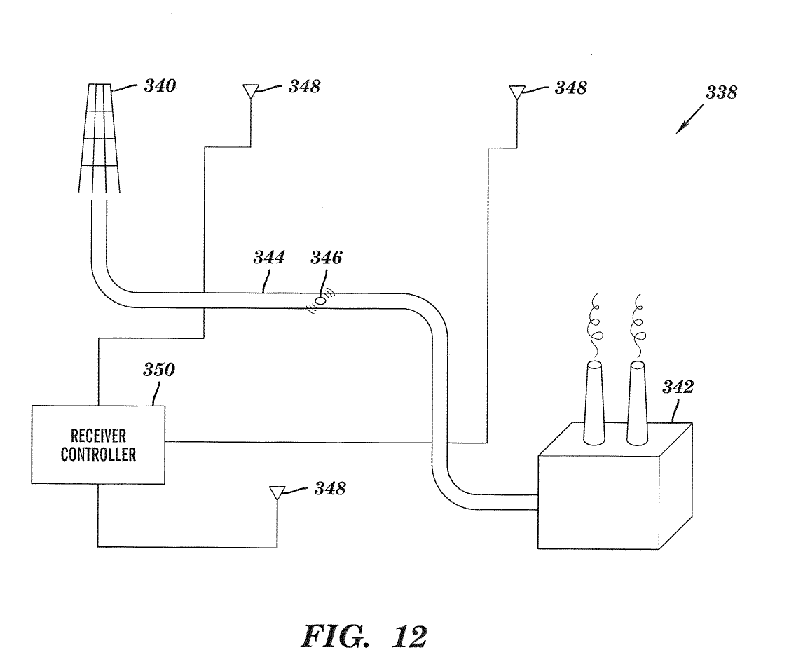

FIG. 12 schematically illustrates another embodiment of a diagnostic system 338. This embodiment illustrates that a diagnostic capsule and its related system do not have to be designed exclusively for people or animals. In this embodiment, a drilling well 340 is coupled to a refinery 342 by a pipeline 344. A diagnostic capsule 346 could be inserted into the pipeline 344 and monitored by a set of at least three receivers 348 which are coupled to a receiver controller 350. While the at least three receivers 348 in this embodiment allow for location techniques such as triangulation, other embodiments could simply have one or more receivers. The diagnostic capsule 346 could be equipped with a variety of sensors, but for this example only, the diagnostic capsule 346 has a target sensor which measures pressure and a diagnostic sensor which is able to image in an oil environment using appropriate wavelengths of light to penetrate the oil. The target pressure sensor could monitor for drops in pressure which might be indicative of a leak in the pipeline. In response to such a target condition, data from the diagnostic sensor (imagery of the pipe area suspected to have a leak) can be transmitted to the receivers 348. The receiver controller 350 can also determine the location of the diagnostic sensor at the time when the suspected leak was determined. A service technician can review the images for the target condition(s) without having to wade through hours of video filled with pipe in good condition. Furthermore, the technician can have a pinpointed location of the problem area. Other embodiments of a diagnostic system could be employed in other piped or pipeline systems.

Having thus described several embodiments of the claimed invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

What is claimed is:

1. A diagnostic capsule, comprising:
a sensor system;
a transmitter; and
a controller which is configured to detect one or more target conditions external to the diagnostic capsule based on target data from the sensor system and enable the transmitter to transmit diagnostic data, wherein the diagnostic data is collected by the sensor system while the one or more target conditions are present.

2. The diagnostic capsule of claim 1, wherein the target data and the diagnostic data are collected by a same sensor in the sensor system.

3. The diagnostic capsule of claim 2, wherein the same sensor is selected from the group consisting of a pH sensor, a temperature sensor, a pressure sensor, a biological sensor, a bacterial sensor, a protein sensor, a chemical sensor, a light sensor, a spectral sensor, a radiation sensor, and an imaging sensor.

4. The diagnostic capsule of claim 1, wherein the sensor system comprises:
a target sensor configured to collect the target data; and
a diagnostic sensor configured to collect the diagnostic data.

5. The diagnostic capsule of claim 4, wherein the target sensor is selected from the group consisting of a pH sensor, a temperature sensor, a pressure sensor, a biological sensor, a bacterial sensor, a protein sensor, a chemical sensor, a light sensor, a spectral sensor, a radiation sensor, and an imaging sensor.

6. The diagnostic capsule of claim 4, wherein the diagnostic sensor is selected from the group consisting of a pH sensor, a temperature sensor, a pressure sensor, a biological sensor, a bacterial sensor, a protein sensor, a chemical sensor, a light sensor, a spectral sensor, a radiation sensor, and an imaging sensor.

7. The diagnostic capsule of claim 4, wherein the diagnostic sensor is further configured to collect diagnostic data irrespective of whether or not the target sensor indicates that the one or more target conditions are present.

8. The diagnostic capsule of claim 7, further comprising a buffer configured to store at least a portion of diagnostic data collected by the diagnostic sensor until the transmitter has been enabled to transmit diagnostic data stored in the buffer.

9. The diagnostic capsule of claim 4, wherein the diagnostic sensor is further configured to collect diagnostic data only when the target sensor indicates that the one or more target conditions are present.

10. The diagnostic capsule of claim 1 further configured for a use selected from the group consisting of an endoscopic capsule, a container inspection capsule, a fluidic inspection capsule, and pipe inspection capsule.

11. A diagnostic system, comprising:
a) a diagnostic capsule, comprising:
i) a sensor system;
ii) a transmitter; and
iii) a controller which is configured to detect one or more target conditions external to the diagnostic capsule based on target data from the sensor system and enable the transmitter to transmit diagnostic data, wherein the diagnostic data is collected by the sensor system while the one or more target conditions are present;
b) at least one receiver configured to receive transmissions from the transmitter; and
c) a receiver controller coupled to the at least one receiver, the receiver controller configured to store transmitted diagnostic data received at the at least one receiver from the diagnostic capsule.

12. The system of claim 11, wherein:
the at least one receiver comprises at least three receivers configured to receive transmissions from the transmitter, each of the at least three receivers located in a separate location; and
the receiver controller is further configured to determine a position of the diagnostic capsule relative to the at least three receivers based on RF telemetry.

13. The system of claim 12, wherein the receiver controller is further configured to store the determined position of the diagnostic capsule relative to the at least three receivers for one or more diagnostic data transmissions received from the transmitter.

14. The system of claim 13, wherein the receiver controller is further configured to store one or more reference points relating a subject being tested by the diagnostic system to the at least three receivers.

15. The system of claim 14, wherein the receiver controller is further configured to determine a location of the diagnostic capsule within the subject based on the RF telemetry determination and the relationship between the subject being tested and the at least three receivers.

16. A method of managing power consumption in a diagnostic capsule, comprising:
enabling a target sensor;
checking for a target condition from the target sensor;
enabling at least one diagnostic capsule subsystem if the target condition is present;
further checking for the target condition; and
disabling the at least one diagnostic capsule subsystem if the target condition is not present.

17. The method of claim 16, wherein the target sensor is selected from the group consisting of a pH sensor, a temperature sensor, a pressure sensor, a biological sensor, a bacterial sensor, a protein sensor, a chemical sensor, a light sensor, a spectral sensor, a radiation sensor, and an imaging sensor.

18. The method of claim 16, wherein the at least one diagnostic capsule subsystem comprises a diagnostic sensor.

19. The method of claim 18, wherein the diagnostic sensor is selected from the group consisting of a pH sensor, a temperature sensor, a pressure sensor, a biological sensor, a bacterial sensor, a protein sensor, a chemical sensor, a light sensor, a spectral sensor, a radiation sensor, and an imaging sensor.

20. The method of claim 16, wherein the at least one diagnostic capsule subsystem comprises a transmitter.

21. The method of claim 16, wherein:
enabling the at least one diagnostic capsule subsystem comprises enabling a diagnostic sensor and enabling a transmitter; and
disabling the at least one diagnostic capsule subsystem comprises disabling the diagnostic sensor and disabling the transmitter.

22. The method of claim 21, wherein the transmitter at least transmits data from the enabled diagnostic sensor while the transmitter and the diagnostic sensor are enabled.

23. The method of claim 16, further comprising enabling a transmitter before checking for the target condition from the target sensor.

24. The method of claim 23, wherein:
enabling the at least one diagnostic capsule subsystem comprises enabling a diagnostic sensor; and
disabling the at least one diagnostic capsule subsystem comprises disabling the diagnostic sensor.

25. The method of claim 24, wherein the transmitter at least transmits data from the diagnostic sensor while the diagnostic sensor is enabled.

26. The method of claim 16, further comprising enabling a diagnostic sensor before checking for the target condition from the target sensor.

27. The method of claim 26, wherein:
enabling the at least one diagnostic capsule subsystem comprises enabling a transmitter; and
disabling the at least one diagnostic capsule subsystem comprises disabling the transmitter.

28. The method of claim 27, wherein the transmitter at least transmits data from the diagnostic sensor while the transmitter is enabled.

29. The method of claim 27, further comprising buffering data from the enabled diagnostic sensor.

30. The method of claim 29, wherein the transmitter at least transmits buffered data from the diagnostic sensor while the transmitter is enabled.

31. The method of claim 16:
wherein enabling the at least one diagnostic capsule subsystem comprises enabling a diagnostic sensor and buffering data from the diagnostic sensor;
wherein disabling the at least one diagnostic capsule subsystem comprises disabling the diagnostic sensor; and
further comprising:
after buffering data from the enabled diagnostic sensor, checking to see of the buffer is at a threshold;
if the buffer is at the threshold, then enabling a transmitter to transmit the buffered data from the diagnostic sensor, then disabling the transmitter.
